(19) United States
(12) Patent Application Publication
(10) Pub. No.: US 2022/0168509 A1
(43) Pub. Date: Jun. 2, 2022

(54) Assembly for a Drug Delivery Device

(71) Applicant: Sanofi, Paris (FR)

(72) Inventors: Michael Jugl, Frankfurt am Main (DE); Stefan Blancke, Frankfurt am Main (DE)

(21) Appl. No.: 17/600,654

(22) PCT Filed: Apr. 1, 2020
(86) PCT No.: PCT/EP2020/059265

§ 371 Date: Oct. 1, 2021

(30) Foreign Application Priority Data
Apr. 4, 2019 (EP) 19305441.8

(51) Int. Cl. A61M 5/24

(52) U.S. Cl. CPC A61M 5/24; A61M 2005/3126; A61M 2205/6072

(57) ABSTRACT

An assembly for a drug delivery device, the assembly comprising: a housing unit, the housing unit comprising a housing for receiving a reservoir and a dispensing mechanism configured to dispense liquid drug content from the reservoir in a dispensing operation, a cap which is releasably connected to the housing unit, the cap, when connected to the housing unit, at least partially covers a distal dispensing end of the assembly, which is uncovered when the cap is disconnected from the housing unit, a first information feature having coded first content, wherein the first information feature is machine-readable to retrieve the first content from the first information feature, a second information feature, wherein the first information feature is visible and/or machine-readable when the cap is connected to the housing unit and removable together with the cap from the housing unit or rendered unreadable when the cap is disconnected from the housing unit.

### CROSS REFERENCE TO RELATED APPLICATIONS

The present application is the national stage entry of International Patent Application No. PCT/EP2020/059265, filed on Apr. 1, 2020, and claims priority to Application No. EP 19305441.8, filed on Apr. 4, 2019, the disclosures of which are incorporated herein by reference.

### TECHNICAL FIELD

The present disclosure relates to an assembly, e.g. for a drug delivery device.

### BACKGROUND

Mobile or carry-on drug delivery devices are often used in emergency situations where there is little time to administer a saving amount of drug to the patient. An alternative use which occurs frequently is self-administration by patients, e.g. medically untrained patients. The unintentional use of an already used drug delivery device can therefore have devastating consequences for the health of the patient. It is therefore desirable to provide drug delivery devices that, due to their appearance, allow a clear distinction between used and unused conditions.

Drug delivery devices are known from the prior art in which it can be judged by intensive investigations of the external appearance whether the drug delivery device has already been used or not. Sometimes the investigator has to inspect visually areas of the device which are remote or tiny features to verify, whether the device has been used or not. However, even such drug delivery devices offer little security. Since an assessment of such drug delivery devices is time-consuming, in particular with a larger number of drug delivery devices, the investigation can quickly fatigue the investigator commissioned with the assessment, which, of course, decisively increases the risk of an incorrect assessment by the investigator.

### SUMMARY

It is an object of the present disclosure to provide an improved assembly for a drug delivery device. Preferably, an assembly should be provided which facilitates the assessment of whether the device has been used potentially or not.

There is provided herein an assembly for a drug delivery device, the assembly comprising a housing unit, the housing unit comprising a housing for receiving a reservoir and a dispensing mechanism configured to dispense liquid drug content from the reservoir in a dispensing operation, a cap which is releasably connected to the housing unit, the cap, when connected to the housing unit, at least partially covers a distal dispensing end of the assembly, wherein the distal dispensing end is uncovered when the cap is disconnected from the housing unit. The cap may be connected to the housing of the housing unit, e.g. by snap features. The assembly further comprises a first information feature having coded first content, wherein the first information feature is machine-readable to retrieve the first content from the first information feature. The assembly may comprise a second information feature, e.g. being machine-readable. The first information feature is visible and/or machine-readable when the cap is connected to the housing unit and removable together with the cap from the housing unit or rendered unreadable when the cap is disconnected from the housing unit. The dispensing mechanism may have two different states, a first state in which the dispensing mechanism is in a ready-to-dispense condition and a second state in which the dispensing mechanism is in a dispensed condition. The second information feature is not visible in the first state and visible in the second state. The assembly may be a drug delivery device. If the reservoir is arranged in the housing, the assembly may be a drug delivery device ready to dispense the content of the reservoir from the device. In the assembly, the reservoir may be received in the housing or not. The reservoir may be a syringe, e.g. a pre-filled syringe.

In the context of the present disclosure, the term “distal dispensing end” may mean an end region of the assembly which is directed towards or designed to abut a target surface, for example a skin portion of the patient, during a dispensing operation. The term “dispensing operation” may refer to the process the dispensing mechanism performs in order to dispense drug from the reservoir of the assembly, expediently via operation of the dispensing mechanism. The drug may be administrated, e.g. via a needle, into the region below the skin of the patient. The dispensing mechanism may be spring driven. The drug delivery device may be a disposable device, which is disposed of after the reservoir has been emptied. The device may be a single use device, which is disposed of after having performed just one single dispensing operation for dispensing one dose.

In the context of the disclosure, the term “ready-to-dispense condition” may refer to a condition of the assembly before the dispensing operation has been commenced or initiated. In this condition, the reservoir may be arranged in the housing or not. In the latter case, the dispensing mechanism performs the movements required for the dispensing operation but does not dispense liquid drug from the device.

In the context of the disclosure, the term “dispensed condition” may refer to a condition after the dispensing operation, preferably after the first or the last dispensing operation which the dispensing mechanism is designed to perform in case a plurality of operations are possible, has taken place. The assembly may be configured to perform just a single dispense operation or a plurality of dispensing operations.

In the context of the disclosure, the term “machine-readable” may refer to a structure or feature which carries information, preferably coded information. For retrieving the information, the structure may be made subject to a reading procedure, preferably using a reader device. For example, the structure may be scanned by the reader device. The reader device may be an optical reader, e.g. utilizing a light source such as a monochromatic light source, preferably on semiconductor basis, such as an LED or a semiconductor laser, for the scanning procedure. The light may be visible light and/or have a specific color, e.g. red. In order to retrieve the information from the structure, a decoding procedure may be necessary. In other words, without the decoding procedure, the information cannot be retrieved. The decoding procedure may be performed either in the reader device or in a processor device operatively connected to the reader device, e.g. a workstation, a server, or a PC. The decoded information may be displayed to the user on a display.

By means of the assembly described further above, an assembly for a drug delivery device is provided which enables a clear and fast detection and/or distinction between a ready-to-dispense condition and a dispensed condition, e.g. due to the visibility or non-visibility of the second information feature and/or due to the first information feature. As the first information feature may be coupled to the cap, the distinction is even possible, when the cap has been removed. Due to the machine-readability of the first information feature, and preferably also of the second information feature, even larger quantities of drug delivery devices can be checked quickly and reliably. This greatly reduces the risk of attempting to use or re-use the assembly in the dispensed condition.

In a preferred embodiment the coded first content of the first information feature may indicate the first state of the dispensing mechanism,

In a preferred embodiment the second information feature may indicate the second state of the dispensing mechanism.

Thus, based on the information content of the information features, it can, for example, be determined whether the assembly has been used already.

In a preferred embodiment the second information feature may have coded second content, wherein the second information feature is preferably machine-readable to retrieve the second content from the second information feature.

Due to the machine-readability of the second information feature even larger quantities of drug delivery devices can be checked quickly and reliably. This greatly reduces the risk of using the drug delivery device in the dispensed condition.

In a preferred embodiment the coded first content of the first information feature may indicate the first state of the dispensing mechanism, and/or the coded second content of the second information feature may indicate the second state of the dispensing mechanism.

Thus, based on the information content of the information features, it can be determined whether the assembly has already been used. For obtaining the respective information, however, a decoding procedure may have to be performed.

In a preferred embodiment the assembly may comprise a movable member. The second information feature may be located on the movable member. The movable member may be in a first position, e.g. relative to the housing, when the dispensing mechanism is in the first state. The movable member may be in a second position, e.g. relative to the housing, when the dispensing mechanism is in the second state. The first position may be different from the second position. The movable member may be configured to move or may move from the first position to the second position when the liquid drug content from the reservoir is dispensed in the dispensing operation and/or after the dispensing operation has been completed. The movable member may be a member of the dispensing mechanism which moves during the dispensing operation and/or a member which moves after the dispensing operation has been completed. In the first position of the movable member, the second information feature may be covered by the, preferably opaque, housing. In the second position, the movable member may be visible, e.g. because it protrudes from the housing or because it is aligned with a window or a window portion provided in the housing.

By providing the second information feature on the movable member, e.g. by attaching the second information feature to the member, it can be ensured that the second information feature is only visible when the dispensing operation has already been completed.

In a preferred embodiment the movable member may be a plunger, which is configured to push or pushes the liquid drug content out of the reservoir towards the distal dispensing end of the assembly in the dispensing operation. The plunger movement may force the liquid drug from the dispensing end. The housing may comprise a window portion. The second information feature and/or the plunger may be visible through the window portion in the second state of the dispensing mechanism and may be not visible through the window portion in the first state of the dispensing mechanism. The plunger may be part of the dispensing mechanism.

If the second information feature is located on the plunger, it is easily visible for the user and/or can be read reliably in the second state.

In a preferred embodiment the movable member is a needle cover. The needle cover may be arranged to cover a needle of the assembly, preferably after the dispensing operation has been completed. The needle cover may be movably connected to the housing and/or be part of the housing unit. In the first state, the needle cover may protrude from the housing. Movement of the needle cover towards the housing may initiate the dispensing operation. The needle cover may be arranged to be moved, e.g. by a spring, relative to the housing and/or into the second position after the dispensing operation has been completed. During dispensing, the needle may be displaced relative to the housing. Preferably the needle cover covers the needle at least after the dispensing operation has been completed and the assembly has been removed from the user. In this position, the needle cover may protrude further from the housing than before the injection has been initiated.

One advantage of a needle cover is to protect both the needle from damage and the user from injuries caused by unintentionally touching the needle. Preferably, the needle cover fulfills this function in the ready-to-dispense condition and/or dispensed condition. By attaching the second information features to the needle cover two effects can be combined. On the one hand, it is achieved that the second information feature is only visible in the second state when the assembly is in the dispensed condition, e.g. due to the needle cover protruding further from the housing after the operation has been completed than in the first state. On the other hand, it is ensured that the user is protected in the second state from needle stick and/or prick injuries. The needle cover preferably does not move into the second position until the assembly is lifted off a target surface (e.g., a skin portion).

In a preferred embodiment the movable member may be a syringe or a cartridge. The syringe may be provided with a needle. The syringe may be a pre-filled syringe. The cartridge may be needle-free and be moved to be in fluid communication with a needle during the dispensing operation.

In a preferred embodiment the movable member may be a trigger member, which is configured to be actuated to trigger or initiate the dispensing operation. In order to trigger the dispensing operation, the trigger member may be moved relative to the housing away from an initial position e.g. into the first position. When a pressure on the trigger member is released, the trigger member may be moved, e.g. back towards and preferably beyond the initial position, into the second position, e.g. by a trigger member spring. In the second position, the trigger member may protrude further from the housing than in the initial position. In that region of the trigger member which is now uncovered as it is no longer covered by the housing, the second information feature may be located. The trigger member may be a trigger button.

In a preferred embodiment the needle cover may act as the trigger member.

In a preferred embodiment the first information feature may be permanently modified when removing the cap, e.g. by detaching the cap from the housing. Thus, after having removed the cap, the coded first content may no longer retrievable from the modified first information feature, preferably even if the cap were re-attached to the housing.

Re-attaching the cap to the housing after the dispensing operation may falsely give the impression that the assembly is still in the ready-to-dispense condition. By permanently modifying the first information feature, for example by destroying the first information feature at least partly, e.g. by tearing a label with the feature on it, or entirely when removing the cap, a wrong assessment of the current state of the device can be avoided. The non-readability may be an (additional) indicator for the state of the assembly.

In a preferred embodiment the assembly may comprise an information carrier connected to the cap and/or the housing unit, wherein the first information feature is located, preferably a least partly or entirely, on the information carrier. The information carrier, e.g. configured ring-like, may establish the connection between the cap and the housing. Thus, it may serve as a connector.

Attaching the first information feature to the information carrier is less expensive than, for example, engraving or etching the first information feature into the surface of the cap and/or the housing unit.

In a preferred embodiment the information carrier may be a sealing.

The information carrier can serve in this way for sealing the interface between cap and housing unit, when the cap is connected to the housing unit.

In a preferred embodiment at least one of the first information feature and the second information feature or both information features may comprise or be a two-dimensional barcode.

Barcodes may assist in eliminating the possibility of human error during information retrieval. A barcode scan is fast and reliable. Moreover, using a barcode system reduces training time for the person who is responsible for checking the drug delivery devices.

In a preferred embodiment at least one of the first information feature and the second information feature or both information features may comprise or be a QR code.

A QR code can be scanned using a smartphone or any other phone with scanning capability. QR codes are versatile and can encode almost all types of data e.g. numeric, alphabets, special and binary. Moreover, they can be scanned extremely fast, which is especially beneficial when a large number of drug delivery devices needs to be scanned.

In a preferred embodiment the assembly may be an auto-injector. The auto-injector may comprise a drive spring which may be part of the dispensing mechanism, e.g. to move the plunger relative to the housing. The auto-injector may be a single dose injector, i.e. a device which is used only once for delivering one single dose. Here, information on the current state of the mechanism is of particular advantage.

Auto-injectors are easy to use and well suited for self-administration by patients or for administration by untrained personnel.

### BRIEF DESCRIPTION OF THE FIGURES

Embodiments will now be described, by way of example only, with reference to the accompanying drawings, in which:

FIG. 1A is a schematic side view of an auto-injector with a cap attached to a housing unit of the auto-injector according to a first embodiment;

FIG. 1B is a schematic side view of the auto-injector of FIG. 1A, with the cap removed from the housing unit;

FIG. 2A is a schematic side view of an auto-injector with a cap attached to a housing unit of the auto-injector according to a second embodiment;

FIG. 2B is a schematic side view of the auto-injector of FIG. 2A, with the cap removed from the housing unit;

FIG. 3A is a schematic side view of a housing unit of an auto-injector according to a third embodiment in a ready-to-dispense condition;

FIG. 3B is a schematic side view of the housing unit of FIG. 3A in a dispensed condition;

FIG. 4A is a schematic side view of a housing unit of an auto-injector according to a fourth embodiment in a ready-to-dispense condition;

FIG. 4B is a schematic side view of the housing unit of FIG. 4A in a dispensed condition;

### DETAILED DESCRIPTION

A drug delivery device, as described herein, may be configured to inject a medicament into a patient. For example, delivery could be sub-cutaneous, intra-muscular, or intravenous. Such a device could be operated by a patient or care-giver, such as a nurse or physician, and can include various types of safety syringe, pen-injector, or auto-injector.

The auto-injector may comprise a drive spring which may be part of a dispensing mechanism, e.g. to move a plunger relative to a housing. The auto-injector may be a single dose injector, e.g. a device which is used only once for delivering one single dose.

The device can include a cartridge-based system that requires piercing a sealed ampule before use. Volumes of medicament delivered with these various devices can range from about 0.5 ml to about 2 ml. Yet another device can include a large volume device (“LVD”) or patch pump, configured to adhere to a patient's skin for a period of time (e.g., about 5, 15, 30, 60, or 120 minutes) to deliver a “large” volume of medicament (typically about 2 ml to about 10 ml).

In combination with a specific medicament, the presently described devices may also be customized in order to operate within required specifications. For example, the device may be customized to inject a medicament within a certain time period (e.g., about 3 to about 20 seconds for auto-injectors, and about 10 minutes to about 60 minutes for an LVD). Other specifications can include a low or minimal level of discomfort, or to certain conditions related to human factors, shelf-life, expiry, biocompatibility, environmental considerations, etc. Such variations can arise due to various factors, such as, for example, a drug ranging in viscosity from about 3 cP to about 50 cP. Consequently, a drug delivery device will often include a hollow needle ranging from about 25 to about 31 Gauge in size. Common sizes are 27 and 29 Gauge.

The delivery devices described herein can also include one or more automated functions. For example, one or more of needle insertion, medicament injection, and needle retraction can be automated. Energy for one or more automation steps can be provided by one or more energy sources. Energy sources can include, for example, mechanical, pneumatic, chemical, or electrical energy. For example, mechanical energy sources can include springs, levers, elastomers, or other mechanical mechanisms to store or release energy. One or more energy sources can be combined into a single device. Devices can further include gears, valves, or other mechanisms to convert energy into movement of one or more components of a device.

The one or more automated functions of an auto-injector may each be activated via an activation mechanism. Such an activation mechanism can include one or more of a button, a lever, a needle sleeve, or other activation component. Activation of an automated function may be a one-step or multi-step process. That is, a user may need to activate one or more activation components in order to cause the automated function. For example, in a one-step process, a user may depress a needle sleeve or cover against their body in order to cause injection of a medicament. Other devices may require a multi-step activation of an automated function. For example, a user may be required to depress a button and retract a needle shield in order to cause injection.

In addition, activation of one automated function may activate one or more subsequent automated functions, thereby forming an activation sequence. For example, activation of a first automated function may activate at least two of needle insertion, medicament injection, and needle retraction. Some devices may also require a specific sequence of steps to cause the one or more automated functions to occur. Other devices may operate with a sequence of independent steps.

Some delivery devices can include one or more functions of a safety syringe, pen-injector, or auto-injector. For example, a delivery device could include a mechanical energy source configured to automatically inject a medicament (as typically found in an auto-injector) and a dose setting mechanism (as typically found in a pen-injector).

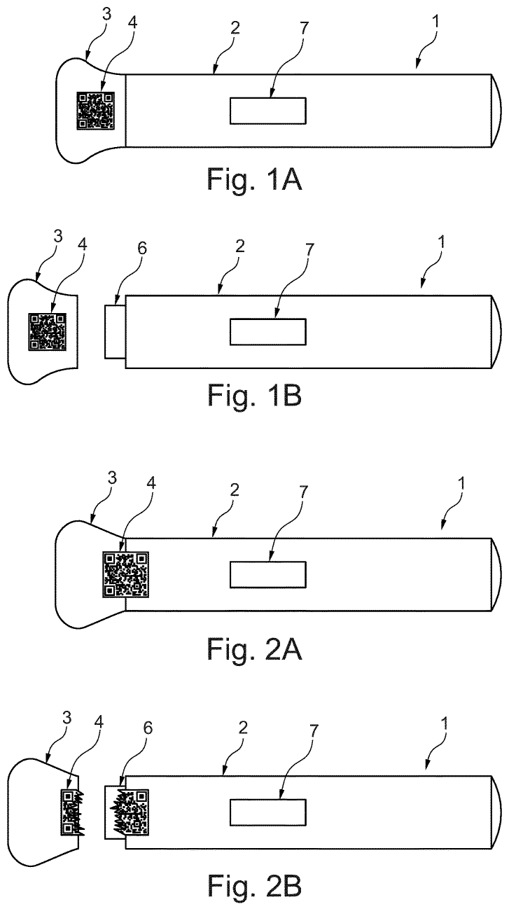

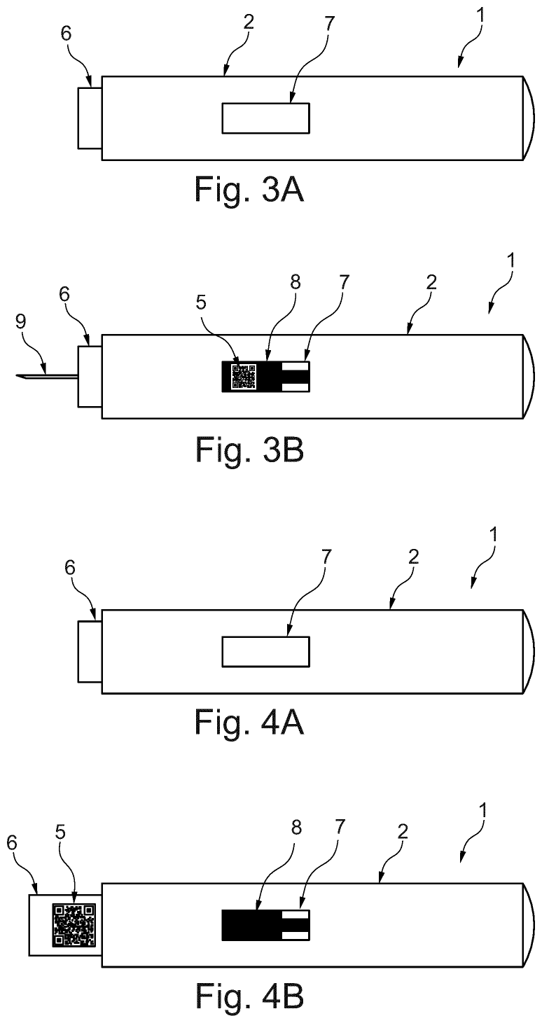

According to some embodiments of the present disclosure, exemplary assemblies 1 for a drug delivery device are shown in FIGS. 1A, 1B, 2A, 2B, 3A, 3B, 4A and 4B. Each of the exemplary assemblies 1 is configured to inject a medicament into a patient's body. The assemblies 1 shown in FIGS. 1A, 1B, 2A, 2B, 3A, 3B, 4A and 4B include a housing unit 2, which typically comprises a housing for receiving a reservoir and a dispensing mechanism configured to dispense liquid drug content from the reservoir in a dispensing operation and/or elements of a dispensing mechanism such as a plunger and/or a spring, a cap 3 (not shown in FIGS. 3A, 3B, 4A, and 4B), which is releasable connected to the housing unit 2, wherein the cap 3, when connected to the housing unit 2 at least partially covers a distal dispensing end of the assembly 1, which is uncovered when the cap 2 is disconnected from the housing, a first information feature 4 (not shown in FIGS. 3A, 3B, 4A, and 4B), having coded first content, wherein the first information feature 4 is machine-readable to retrieve the first content from the first information feature 4, wherein the coded first content of the first information feature 4 indicates a first state of the dispensing mechanism in which the dispensing mechanism is in a ready-to-dispense condition, and a second information feature 5 (not shown in FIGS. 1A, 1B, 2A, and 2B), wherein the second information feature 5 indicates a second state of the dispensing mechanism in which the dispensing mechanism is in a dispensed condition. Furthermore it is possible that the second information feature 5 having coded second content, wherein the second information feature 5 is machine-readable to retrieve the second content from the second information feature 5 and the coded second content indicates the second state of the dispensing mechanism in which the dispensing mechanism is in the dispensed condition. In addition to this, the housing unit 2 of the shown assemblies 1 comprise a window portion 7. The window portion 7, with the exception of the embodiment of FIGS. 3A and 3B, may also be dispensed with.

For retrieving the information or content from the respective machine-readable information feature, the structure may be or has to be made subject to a reading procedure, preferably using a reader device. For example, the structure may be scanned by the reader device. The reader device may be an optical reader, e.g. utilizing a light source such as a monochromatic light source, preferably on semiconductor basis, such as an LED or a semiconductor laser, for the scanning procedure. The light may be visible light and/or have a specific color, e.g. red. In order to retrieve the information from the structure, a decoding procedure may be necessary. In other words, without the decoding procedure, the information cannot be retrieved. The decoding procedure may be performed either in the reader device or in a processor device operatively connected to the reader device, e.g. a workstation, a server, or a PC. The decoded information may be displayed to the user on a display.

In the shown examples of FIGS. 1A, 1B, 2A and 2B, the first information feature 4 is a QR code, which is located on an information carrier. The information feature may be provided on a label or sticker, which is attached to the information carrier, or may be integrated into the information carrier. The information carrier may be a rigid part or conformable to the outer surface of the cap and/or the housing unit. However, other data representation, for example other variants of, preferably multi-dimensional, e.g. two-dimensional, barcodes, may be suitable for the information feature. The information carrier, e.g. configured ring-like, may establish the connection between the cap 3 and the housing 2. Thus, it may serve as a connector. Alternatively or additionally, the information carrier may be a sealing. In this case the information carrier can serve for sealing the interface between cap 3 and housing unit 2, when the cap 3 is connected to the housing unit 2.

FIG. 1A and FIG. 1B show a schematic side view of the assembly 1 according to a first embodiment, wherein the first information feature 4 is located on a surface of the cap 3. In FIG. 1A the cap 3 is attached to a housing unit 2, therefore the first information feature 4 is visible and indicates the first state. In FIG. 1B the cap 3 is removed from the housing unit 2. If the cap 3 is set aside by a user, the remaining components of the assembly 1 no longer contain any information that the dispensing mechanism of the assembly 1 is in the first state.

FIG. 2A and FIG. 2B show a schematic side view of the assembly 1 according to a second embodiment, wherein the first information feature 4 is located partially on the surface of the cap 3 and on a surface of the housing unit 2. In FIG. 2A the cap 3 is attached to a housing unit 2, therefore the first information feature 4 is visible and indicates the first state. In FIG. 2B the cap 3 is removed from the housing unit 2. Due to the removing of the cap 3 from the housing unit 2, the first information feature 4 is destroyed permanently such that, after having removed the cap 3, the coded first content is no longer retrievable from the destroyed first information feature 4. If the information carrier extends along an intermediate region between the cap and the housing unit, e.g. circumferentially (not explicitly shown), the sealing of an interface between the cap and the housing unit may be improved.

In the previously described embodiments, the information carrier is preferably a conformable element preferably a sticker or label applied to the cap and/or the housing unit.

In another, not depicted embodiment, the information carrier may be a rigid part, e.g. a sleeve, or an elastic part, e.g. a seal, such as of rubber. The information carrier may be arranged between the cap and the housing or housing unit, e.g. clamped, when the cap is directly connected to the housing or the housing unit. The information carrier may be connected to the cap such as via a positive connection, e.g. a snap fit, as well as to the housing unit, such as via a positive connection, e.g. a snap fit. The connections between information carrier and housing unit and between cap and information carrier may be designed to be incompatible. That is to say, the cap, preferably, cannot be attached to the housing unit directly without the intermediate information carrier. The information carrier may have to be detached from the housing unit for achieving operability of the device. The connection between the information carrier and the housing and/or between the cap and the information carrier may be designed to be releasable only by modifying at least one of the connection features which engage with each other to establish the connection, such that, after the connection has been released it cannot be established again. Thus, the information carrier and/or the information feature may be indicative for the drug contained in the reservoir. A label or sticker may be provided with the information feature which may, preferably exclusively, be arranged on and/or connected to the information carrier.

FIG. 3A and FIG. 3B show a schematic side view of the housing unit 2 according to a third embodiment. As in a fourth embodiment, shown in FIG. 4A and FIG. 4B, the housing unit 2 of the third embodiment comprises the needle 9, for delivering liquid drug content from the reservoir in the dispensing operation, a needle cover 6, which protectively encloses the needle 9, and a plunger 8, which pushes the liquid drug content out of the reservoir to the distal dispensing end of the assembly 1 in the dispensing operation. The plunger is part of the dispensing mechanism.

FIG. 3A shows the third embodiment in the first state of the dispensing mechanism, where the dispensing mechanism is in a ready-to-dispense condition. As in the fourth embodiment in the first state of the dispensing mechanism, shown in FIG. 4A, a second information feature 5 is not visible in the first state, i.e. when the dispensing operation has not been performed and/or initiated. However, the second information feature 5 in the second state is visible in the third embodiment, shown in FIG. 3B, and the fourth embodiment, shown in FIG. 4B. Consequently, the second feature 5 is only visible when the dispensing mechanism is in the dispensed condition after the dispensing operation.

In the embodiments shown in the FIGS. 3A, 3B, 4A and 4B, the second information feature 5 is located on a movable member 6, 8, wherein the movable member 6, 8 is in a first position, e.g. relative to the housing (as shown in FIGS. 3A and 4A), when the dispensing mechanism is in the first state, wherein the movable member 6, 8 is in a second position, e.g. relative to the housing (as shown in FIGS. 3B and 4B), when the dispensing mechanism is in the second state, and wherein the movable member 6, 8 moves from the first position to the second position when the liquid drug content from the reservoir is dispensed in the dispensing operation or has been dispensed from the reservoir.

In the third embodiment shown in FIGS. 3A and 3B the movable member is the plunger 8. As shown in FIG. 3B the second information feature 5 and the plunger 8 are visible through the window portion 7 in the second state of the dispensing mechanism and, as shown in FIG. 3A, not visible through the window portion 7 in the first state of the dispensing mechanism. In this case, the reservoir, e.g. a syringe or a cartridge, may be securely retained in the housing unit and may not move during the dispensing operation which moves the plunger relative to the reservoir. Alternatively, the movable member may be the reservoir, e.g. a syringe or a cartridge, which moves for dispensing its content e.g. relative to the plunger which may be static. The syringe may be provided with the needle 9. The syringe may be a pre-filled syringe. The cartridge may be needle-free and be moved into fluid communication with the needle 9 during the dispensing operation.

In the fourth embodiment shown in FIGS. 4A and 4B, the movable member is the needle cover 6, which is arranged to cover the needle 9 (not visible in FIGS. 4A and 4B) of the assembly 1 after the dispensing operation has been completed and/or before it is commenced. The needle cover 6 is movably connected to the housing and/or is part of the housing unit 2.

After activation of the dispensing mechanism, e.g. via a trigger member, the dispensing of the liquid drug content takes place via operation of the dispensing mechanism. After the dispensing operation has been completed and/or after the assembly is lifted off a target surface (e.g., a skin portion), the needle cover 6 moves distally, e.g. on account of a spring, from a first position as shown in FIG. 4A, in which the second information feature is not visible, to a second position, as shown in FIG. 4B, in which the second information feature 5 is visible on the needle cover 6. For example the needle cover 6 may act as the trigger member. In this case, the dispensing operation is triggered when the needle cover 6 is pressed onto the target surface and the housing unit is moved relative to the needle cover. This movement may actuate the dispensing mechanism to conduct the dispensing operation. Here, the needle cover 6 may protrude from the housing in the first state. A movement of the needle cover 6 towards the housing initiates the dispensing operation and/or allows the needle to penetrate the target tissue or skin. The needle cover 6 is arranged to be moved, e.g. by a spring, relative to the housing and/or into the second position after the dispensing operation has been completed. During dispensing, the needle 9 may be displaced relative to the housing or be stationary. As shown in FIG. 4B, the needle cover 6 covers the needle 9 at least after the dispensing operation has been completed and the assembly has been removed from the user. In this position, the needle cover 6 protrudes further from the housing than before the injection has been initiated thereby revealing the second information feature.

The needle cover, however, does not have to act as trigger member as has been described above to initiate the dispensing operation. Rather, the assembly/device may comprise a separate trigger member, e.g. a trigger button, movable relative to the housing unit by the user, e.g. pressable, to trigger the dispensing operation. In order to trigger the dispensing operation, the trigger member may be moved relative to the housing away from an initial position e.g. into the first position. When a pressure, e.g. exerted by the user, on the trigger member is released, the trigger member may be moved back towards and preferably beyond the initial position into the second position, e.g. by a trigger member spring. In the second position, the trigger member may protrude further from the housing than in the initial position. In that region which is now uncovered, the second information feature 5 may be located (not illustrated). Accordingly, the trigger member may act as a movable member with the second information feature (not illustrated). The trigger member may be provided with an information feature in addition to or as an alternative to the needle cover. Thus, there may be just one or a plurality of “second” information features on elements which move after initiation of the dispensing operation, during and/or after completion of the dispensing operation to indicate that the assembly is in the dispensed condition.

In the above-described embodiments the second information feature 5 may be a QR code. However, the second information feature 5 may also comprise other kinds of, preferably machine-readable, representation of data, for example other variants of multi-dimensional, e.g. two-dimensional, barcodes.

It should be noted that the third embodiment, as shown in FIGS. 3A and 3B, in particular the arrangement of the second information feature 5, can be combined with both the first and the second embodiment, as shown in FIGS. 1A, 1B, 2C, and 2B, in particular with the arrangement of the first information feature 4. The same holds for the fourth embodiment, as shown in FIGS. 4A and 4B. Moreover it is also possible to add an additional second information feature 5 as in the third embodiment on the plunger 8 of the fourth embodiment as shown in FIG. 4A and FIG. 4B. Also, features disclosed in the introductory section before the description of the embodiments in conjunction with the drawings may be combined with features described in conjunction with the exemplary embodiments shown in the drawings, of course.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders.

Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An example of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

1-15. (canceled)

16. An assembly for a drug delivery device, the assembly comprising:
a housing unit comprising a housing for receiving a reservoir and a dispensing mechanism configured to dispense a liquid drug content from the reservoir in a dispensing operation;
a cap that is releasably connected to the housing unit, the cap, when connected to the housing unit, at least partially covering a distal dispensing end of the assembly that is uncovered when the cap is disconnected from the housing unit;
a first information feature having a coded first content and being machine-readable for retrieval of the coded first content from the first information feature; and
a second information feature,
wherein the first information feature is visible and/or machine-readable when the cap is connected to the housing unit,
wherein the first information feature is removable together with the cap from the housing unit or rendered unreadable when the cap is disconnected from the housing unit,
wherein the dispensing mechanism has a first state in which the dispensing mechanism is in a ready-to-dispense condition and a second state in which the dispensing mechanism is in a dispensed condition, and
wherein the second information feature is not visible in the first state and visible in the second state.

17. The assembly according to claim 16, wherein the coded first content of the first information feature indicates the first state of the dispensing mechanism, and wherein the second information feature indicates the second state of the dispensing mechanism.

18. The assembly according to claim 16, wherein the second information feature has a coded second content, and wherein the second information feature is machine-readable for retrieval of the coded second content from the second information feature.

19. The assembly according to claim 18, wherein the coded first content of the first information feature indicates the first state of the dispensing mechanism, and wherein the coded second content of the second information feature indicates the second state of the dispensing mechanism.

20. The assembly according to claim 16, further comprising a movable member, wherein the second information feature is located on the movable member, wherein the movable member is in a first position when the dispensing mechanism is in the first state, wherein the movable member is in a second position when the dispensing mechanism is in the second state.

21. The assembly according to claim 20, wherein the movable member is configured to move from the first position to the second position during the dispensing operation and/or after completion of the dispensing operation.

22. The assembly according to claim 21, wherein the movable member comprises a plunger that pushes the liquid drug content out of the reservoir towards the distal dispensing end of the assembly during the dispensing operation.

23. The assembly according to claim 22, wherein the housing comprises a window portion, and wherein the second information feature and the plunger are visible through the window portion in the second state of the dispensing mechanism and not visible through the window portion in the first state of the dispensing mechanism.

24. The assembly according to claim 21, wherein the movable member comprises a needle cover that is arranged to cover a needle of the assembly.

25. The assembly according to claim 21, wherein the movable member comprises a syringe.

26. The assembly according to claim 21, wherein the movable member is a trigger member, which, when actuated, triggers the dispensing operation.

27. The assembly according to claim 21, wherein the first information feature is permanently modified when removing the cap.

28. The assembly according to claim 27, wherein after having removed the cap, the coded first content is no longer retrievable from the modified first information feature.

29. The assembly according to claim 16, further comprising an information carrier connected to the cap and/or to the housing unit, wherein the first information feature is located on the information carrier.

30. The assembly according to claim 29, wherein the information carrier comprises a sealing.

31. The assembly according to claim 16, wherein at least one of the first information feature and the second information feature comprises a two-dimensional barcode.

32. The assembly according to claim 16, wherein at least one of the first information feature and the second information feature comprises a QR code.

33. The assembly according to claim 16, wherein the assembly comprises an auto-injector.

34. The assembly according to claim 16, further comprising the reservoir, that comprises a liquid drug.
